(19) United States
(12) Patent Application Publication
(10) Pub. No.: US 2012/0059413 A1
(43) Pub. Date: Mar. 8, 2012

(54) THREAD, ESPECIALLY SURGICAL THREAD, AN IMPLANT COMPRISING THE THREAD AND ALSO A PROCESS FOR PRODUCING THE THREAD AND THE IMPLANT

(75) Inventors: Marta Gonzàlez Calero, Rubi (ES); Erich Odermatt, Schaffhausen (CH)

(73) Assignees: B. BRAUN SURGICAL S.A., Spain; AESCULAP AG, Tuttlingen/Donau (DE)

(21) Appl. No.: 13/197,064

(22) Filed: Aug. 3, 2011

(30) Foreign Application Priority Data
Aug. 6, 2010 (DE) 10 2010 034 471.0

(51) Int. Cl. A61B 17/04; B05D 7/00

(52) U.S. Cl. 606/228; 427/2.31

(57) ABSTRACT

A surgical thread includes an elongate, preferably braided, thread body and a coating surrounding the thread body at least partially and preferably completely, wherein the thread body includes polyethylene and/or polypropylene and the coating consists of a resorbable material and optionally additives.

### RELATED APPLICATION

This application claims priority of German Application No 10 2010 034 471.0, filed Aug. 6, 2010.

### TECHNICAL FIELD

This disclosure relates to a thread, especially a surgical thread, an implant comprising the thread and also a production process for the thread and the implant.

### BACKGROUND

Ever since surgical sutures were first used, the suture material has undergone constant further development. The natural materials originally used have come to be largely replaced by synthetic suture materials. Depending on the field of surgery, the suture materials used are resorbable or nonresorbable or else optionally partially resorbable.

While nonresorbable suture materials are typically used for vascular, especially cardiovascular, and orthopedic applications, resorbable suture materials are mainly used in gastrointestinal, gynecological and plastic surgery, especially for skin stretching, and for approximation of soft tissue.

Nonresorbable suture materials suitable in principle can consist of ultrahigh molecular weight polyethylene (UHMWPE). Suture materials of this type are notable for very high tensile strengths. A disadvantage is the smooth/slippery surface of suture materials made of UHMWPE. This slipperiness can impair the knot security of a suture and thus endanger wound closure. To nonetheless be able to ensure secure wound closure, the surgeon frequently has to place two or more knots, in some instances even more than six knots, on top of each other. This means an increased introduction of foreign material into the patient, heightening the risk of inflammatory reactions and tissue erosions. Apart from that, the superposition of several knots to ensure adequate knot security is an inconvenient procedure for the surgeon and also in principle susceptible to error. When knots are placed too loosely, this can cause wound dehiscence. When the knots are placed too firmly, by, contrast, outcomes may be cosmetically unsatisfactory owing to tissue necrosis.

EP 1 543 848 A1 describes technical teachings that proceed in another direction. The suture material proposed has a core-sheath construction where the core includes a bioabsorbable polymer. The sheath is a braid and comprises a nonabsorbable yarn and a bioabsorbable yarn. The suture material thus consists predominantly of resorbable material, but this is in turn disadvantageous for its tensile strength and hence mechanical stability.

It could therefore be helpful to provide a thread which is improved in respect of wound closure and especially the security of wound closure. In contradistinction to existing threads of the type in question, it could be helpful to provide a thread that is especially notable in that less foreign material has to be introduced into the body of a patient to ensure secure wound closure.

### SUMMARY

We provide a surgical thread including an elongate, optionally braided, thread body and a coating at least partially surrounding the thread body, wherein the thread body comprises polyethylene and/or polypropylene and the coating consists of a resorbable material and, optionally, additives.

We also provide an implant in the form of a surgical suture material including the above-mentioned thread.

We further provide a process for producing the above-mentioned thred including at least partially coating the polyethylene and/or polypropylene with the resorbable material and optionally the additives.

Lastly, we provide a process for producing the above-mentioned implant including at least partially coating the polyethylene and/or polypropylene with the resorbable material and optionally the additives.

### BRIEF DESCRIPTION OF THE DRAWING

FIG. 1 is a graph showing capillarity of coated and uncoated threads.

### DETAILED DESCRIPTION

We provide a thread, especially a medical or surgical thread, which comprises an elongate thread body and a coating surrounding the thread body at least partially, preferably completely, i.e. all-over. The thread body comprises polyethylene and/or polypropylene. The coating consists of a resorbable material and optionally of additives.

Preferably, the thread body comprises polyethylene. It is particularly favorable for the thread body to comprise polyethylene and/or polypropylene, preferably polyethylene.

The polyethylene and/or polypropylene are preferably a polyethylene homopolymer and/or polypropylene homopolymer.

The polyethylene and/or polypropylene may be a polyethylene copolymer and/or polypropylene copolymer. The copolymer can be more particularly present as random copolymer, alternating copolymer, block copolymer/segmented copolymer and/or as graft copolymer. The copolymer may further have an isotactic, syndiotactic or atactic structure.

A copolymer is a polymer which, in addition to ethylene and/or propylene, includes at least one further monomer unit. Therefore, the term copolymer shall also comprehend, for example, terpolymers, tetrapolymers and the like.

The polyethylene is preferably selected from the group consisting of high density polyethylene (HDPE), low density polyethylene (LDPE), high molecular weight polyethylene (HMWPE), ultrahigh molecular weight polyethylene (UHMWPE), copolymers thereof and mixtures, especially blends, thereof.

Particularly preferably, the polyethylene is an ultrahigh molecular weight polyethylene (UHMWPE). This is a particularly advantageous way of providing a medically optimal mechanical stability, especially linear tensile strength, for the thread.

To further improve the mechanical stability of the thread, it may be provided that the polyethylene and/or polypropylene are present in a crosslinked state, especially in a chemically and/or physically crosslinked state. For example, the polyethylene and/or polypropylene can be present in a crosslinked state as a consequence of a peroxide treatment. Physical crosslinking of polyethylene and/or polypropylene can be effected for example using irradiation, especially ionizing irradiation. For example, the polyethylene, and/or polypropylene, can be present in a crosslinked state as a result of a treatment with γ-rays, β-rays, x-rays, ultraviolet rays, neutron beam rays, proton beam rays and/or electron beam rays.

Particularly preferably, the polyethylene is a crosslinked ultrahigh molecular weight polyethylene (UHMWPE). Concerning suitable methods of crosslinking, they may be cross-linked as described above.

The polyethylene is preferably an ultrahigh molecular weight polyethylene (UHMWPE) having an average molecular weight between about 10⁴ and about 10⁷ g/mol, especially about 10⁵ and about 10⁶ g/mol.

The thread body is particularly preferably a multifil, especially braided or twisted, thread body, especially a multifilament yarn. In this way, the advantages of a multifil thread such as flexibility and tying properties for example can also be realized for the thread. The thread body is preferably a braided thread body. It can be especially provided for the thread body to be configured as a braid with a core. In specific examples, individual filaments of a multifil thread body can be bonded, especially melted, together.

In one example, the thread body has a monofil configuration.

It can further be provided that the thread body is present as a pseudo monofilament.

Particularly preferably, the coating of the thread is a nontextile coating. Such a coating provides for example a distinct reduction in the capillarity and the attendant potential infection risk for a thread having a multifil thread body. A nontextile coating also has the advantage that it can be applied to the thread body, or produced together with the thread body, using relatively simple techniques. Appropriate techniques are more particularly described hereinbelow.

However, in principle, it is possible for the coating to be a textile coating. In this case for example, the coating can be fibrous, especially braided. Alternatively, the coating can also be present, for example, as a textile mesh which surrounds the thread body at least partially, preferably completely.

Preferably, the coating or to be more precise the coating surface has a certain roughness. More particularly, fine hair cracks or fissures can be formed on the surface of the coating in the event of the thread being subjected to a load, increasing the thread's coefficient of friction. An increased coefficient of friction for the thread means increased knot security and, hence, a secure wound closure. Improved friction on the thread more particularly means that fewer knots are needed to bring about secure wound closure. This means reduced material requirements and more particularly a lower input of foreign material into the body of a patient.

The resorbable material of the coating may in principle be a single resorbable material or alternatively a mixture of different resorbable materials. Suitable resorbable materials are more particularly described hereinbelow.

In general, the resorbable material is a polymer, preferably a synthetic polymer. The polymer may be a copolymer in particular. For example, the resorbable material may be present in the form of a random copolymer and/or block copolymer such as a di- and/or triblock copolymer for example.

Particularly suitably, the resorbable material is a polyhydroxyalkanoate or a polyhydroxyalkanoate mixture. The resorbable material is preferably a polyhydroxyalkanoate with two or more different hydroxyalkanoate units.

Preferably, the resorbable material is a polymer comprising at least one monomer unit selected from the group consisting of glycolide, lactide, 3-hydroxybutyrate, 4-hydroxybutyrate, trimethylene carbonate, para-dioxanone, ε-caprolactone and mixtures thereof.

The resorbable material is preferably a polymer selected from the group consisting of polyglycolide, polylactide, poly-3-hydroxybutyrate, poly-4-hydroxybutyrate, polytrimethylene carbonate, poly-para-dioxanone, poly-ε-caprolactone, copolymers thereof and mixtures, especially blends, thereof.

The resorbable material is more preferably a copolymer based on glycolide and lactide, preferably in a weight ratio ranging from about 9:1 to about 1:9 and especially from about 7:3 to about 3:7. Further preferred copolymers comprise ε-caprolactone, trimethylene carbonate and a glycolide or glycolide and ε-caprolactone. An especially preferred copolymer is a terpolymer, in particular triblock terpolymer, made of glycolide, trimethylene carbonate and ε-caprolactone. Such a terpolymer is commercially available under the trademark MONOSYN®.

In one example, the resorbable material is a biopolymer or a biopolymer mixture. The biopolymer can be a naturally occurring polymer and/or a synthetic, especially recombinantly produced, biopolymer. Biopolymers can be selected from the group consisting of proteins such as, for example, extracellular, especially fibrous, proteins or connective tissue proteins, polysaccharides such as, for example, oxidized polysaccharides, mucopolysaccharides and glycosaminoglycans, derivatives thereof, salts thereof and mixtures thereof. The resorbable material can be, for example, a biopolymer selected from the group consisting of collagen, gelatin, elastin, reticulin, fibronectin, fibrilin, starch, amylose, amylopectin, dextran, chitosan, hyaluronic acid, heparin, heparan sulfate, chondroitin 4-sulfate, chondroitin 6-sulfate, dermatan sulfate, keratan sulfate, cellulose, methylcellulose, hydroxymethylcellulose, hydroxyethylcellulose, hydroxypropylcellulose, hydroxybutylcellulose, hydroxyethylmethylcellulose, hydroxypropylmethylcellulose, carboxymethylcellulose, silk, salts thereof, derivatives thereof and mixtures thereof.

The resorbable material may more particularly be a mixture of the materials described above.

The coating on the thread can consist exclusively of the resorbable material.

However, it may be preferable for the coating to comprise the resorbable material and additives. The additives make it possible for the thread to be endowed with medically, especially therapeutically, advantageous properties.

It may further be provided for the thread body to also include additives. Alternatively, additives can be present exclusively in the thread body of the thread.

The expression “additives” comprises not only a single additive, but also a mixture of two or more different additives. The additives may further be polymeric additives or nonpolymeric, i.e., low molecular weight, additives, for example.

Preferred additives may be medical/pharmaceutical actives. Advantageous additives can be selected, for example, from the group consisting of antimicrobial, especially antibiotic, actives, disinfecting actives, active promoters of wound healing, anti-inflammatory actives, analgesic actives, cellular growth factors, cellular differentiation factors, cellular recruitment factors, cellular adhesion factors, derivatives thereof, salts thereof and mixtures thereof.

Preferred antimicrobial actives can be organic compounds and/or metals, especially metal salts such as metal oxides for example. Examples of antimicrobially active organic compounds can be selected from the group consisting of polyhexamethylenebiguanide, chlorhexidine, derivatives thereof, salts thereof and mixtures thereof. Advantageous antimicrobially active metals and metal salts can be selected from the group consisting of copper, silver, gold, salts, especially oxides, thereof and mixtures thereof.

Particularly preferably, optional additives include salts, especially organic salts, preferably fatty acid salts and more preferably alkali metal fatty acid salts and/or alkaline earth metal fatty acid salts. For example, the additives may include magnesium and/or calcium fatty acid salts, especially magnesium and/or calcium stearate.

In a more advanced example, the additives, preferably in the form of fatty acid salts, account for a proportion between about 0.5 wt. % and about 5 wt. %, especially about 1.5 wt. % and about 2.5 wt. % and preferably about 1 wt. % and about 2 wt. %, based on the total weight of the thread.

The coating itself preferably accounts for a proportion between about 1 wt. % and about 9 wt. %, especially about 2 wt. % and about 6 wt. %, preferably about 3 wt. % and about 6 wt. % and more preferably about 2.5 wt. % and about 4.5 wt. %, based on the total weight of the thread.

In a further example, the coating has a layer thickness between about 1 μm and about 100 μm, especially about 3 μm and about 80 μm and preferably about 5 μm and about 50 μm.

The coating is preferably compact and, more particularly, sealing.

The thread further preferably has a linear tensile strength between about 10 N and about 250 N, especially about 20 N and about 210 N and more preferably about 30 N and about 190 N. Linear tensile strength is the force in newtons [N] needed to break the thread, as measured on the straightened thread.

In a further advantageous example, the thread has a knot breaking strength between about 5 N and about 140 N, especially about 15 N and about 120 N and preferably about 20 N and about 100 N. Knot breaking strength is the force in newtons [N] needed to break the knotted thread, as measured in the knot of the thread.

The flexibility of the thread is preferably between about 1 mN and about 50 mN, especially about 3 mN and about 40 mN and preferably about 5 mN and about 30 mN.

Preferably, the thread has a core-sheath construction where the core is formed by the thread body and the sheath is formed by the coating. More particularly, the thread can be configured as a coextrusion thread or sheath extrusion thread.

The thread can be of monofil or multifil configuration. The thread is preferably a pseudo monofil thread. In other words, it can be preferable for the thread body itself to be present as multifilament. The thread body can be present as a braided structure in particular. The thread body is preferably formed as a braid with a core.

The thread may be present as a sterilized and preferably end-itemed, especially cut-to-length, thread.

Particularly preferably, the thread is configured as surgical suture material.

The thread may be attached to one or more, especially two, surgical needles.

We further provide an implant, especially a medical/surgical implant, comprising at least one, especially one, thread, especially one medical/surgical thread, having an elongate, preferably braided, thread body and a coating surrounding the thread body at least partially, preferably completely, wherein the thread body comprises polyethylene and/or polypropylene and the coating consists of a resorbable material and optionally additives.

The implant is preferably a textile implant. More particularly, the implant may be executed as textile mesh, for example as hernia mesh, urinary incontinence mesh or prolapse mesh. In addition, the implant may in principle also comprise other textile implants such as, for example, vascular prostheses, stents, stent linings or the like.

However, it is particularly preferable for the implant to be configured as a surgical suture material.

Concerning further features and advantages, especially in relation to the thread, the thread body and/or the coating, express reference is made to the description above.

We also provide processes for producing a thread, especially a medical/surgical thread, or an implant, especially a medical/surgical implant, wherein an elongate, preferably braided, thread body comprising polyethylene and/or polypropylene is coated with a resorbable material and optionally additives.

The coating of the thread body may only be partial. Preferably, however, the thread body is coated completely, i.e., all-over, with the resorbable material and optionally the additives.

The coating can be effected using a core-sheath extrusion. This is generally accomplished via a coextrusion of the thread body and of the coating. In the process, a core-sheath construction can be realized. For example, a bicomponent extrusion can be carried out to produce the thread/implant.

Alternatively, the thread body may be coated with the resorbable material and optionally the additives using a sheathing extrusion. In this example, the thread body can be used as monofilament or multifilament, especially multifilament yarn. When a multifil thread body is used, this can be used to produce pseudo monofil threads having the properties described above.

Further, the coating of the thread body may be effected using a soaking, wetting, dipping, spraying, brushing and/or calendering technique. Depending on the particular coating technique used, it is advantageous to use the resorbable material in the form of a dispersion, suspension, solution or melt.

The above-described techniques for coating the thread body are relatively simple and, more particularly, economical to carry out and moreover permit not only a partial coating but also a complete, i.e., all-over, coating of the thread body.

As mentioned, we provide the option of additionally coating the thread body with additives. These additives for coating the thread body may already be present in the resorbable material. For this, dispersions, suspensions, solutions or melts of the resorbable material can be provided with the additives, for example. This approach has the advantage of allowing a uniform/homogeneous distribution of the additives in the resorbable material and so with particular advantage a similarly uniform/homogeneous distribution of the additives in the coating of the final thread is obtainable.

Alternatively, the optional additives can also be introduced into the coating only in a subsequent treatment step of the thread.

Concerning further features and advantages of the process, especially in relation to the thread, the thread body and/or the coating, express reference is made to the description above.

Finally, we provide for the use of a thread comprising an elongate, preferably braided, thread body and a coating surrounding the thread body at least partially and preferably completely, wherein the thread body comprises polyethylene and/or polypropylene and the coating consists of a resorbable material and optionally additives, for producing an implant, especially a surgical implant, preferably a surgical suture material.

To avoid unnecessary repetition, concerning further features and advantages of the thread, especially in relation to its thread body and/or coating, the preceding description is likewise referenced in its entirety.

At this point, selected advantages will be summarized as follows:

The thread is particularly advantageous in that, first, the material constitution of the thread body provides it with a very high basic mechanical stability which especially facilitates the passage through tissue and placement of knots. This supports the achievement of secure wound closure.

Second, the coating on the thread provides it with improved friction properties which lead to a distinct improvement in knot security, especially knot breaking strength. More particularly, the improved friction of the thread requires fewer knots to be placed to produce a secure wound closure which is riskless for the patient. As a result, less thread material and, hence, less foreign material is introduced into the body of the patient. As a result, the risk of undesirable secondary reactions such as rejection reactions or tissue erosions, which can be attributable to the shear volume of suture material knots, is distinctly minimized.

The fact that fewer knots have to be placed to ensure secure wound closure significantly simplifies handling of the thread for the physician/surgeon involved. In addition, the propensity to mistakes in placing the knots and, hence, in bringing about wound closure can be distinctly reduced.

A further advantage of the thread is that, when the thread body is multifil, especially braided, the coating ensures that the thread shows distinctly reduced capillarity. This makes it possible to take advantage of the inherent virtues of a multifil thread such as flexibility and tying properties, for example, while at the same time incurring a distinctly reduced risk of capillary-based infections.

Further features and advantages become apparent from the ensuing description of representative, non-limiting examples. Individual features may here be actualized each on its own or in combination with each or one another. The examples described serve for elucidation and better understanding and are not in any way to be understood as restrictive.

### EXAMPLES

### 1. Material

The material used was a UHMWPE fiber having an approximate molecular weight of 2.5×10⁶ g/mol (Dyneema®).

### 2. Coating

The fibers to be coated were led from one spool over a further spool to endow the fibers with desired speed and tension. The fibers were subsequently routed into a coating bath and led over a further spool through a heating duct. The length of the fibers within the heating duct was adjustable via mobile spools, and so parameters such as, for example, fiber tension and residence time of the fibers in the heating duct were also adjustable. The dried fibers were led out of the heating duct and wound up on a further spool.

The fibers thus coated had a coating proportion of about 2% by weight, based on the total weight of the coated fiber.

The fibers were coated using the following solutions:
1^{st} solution containing a copolymer based on glycolide (54 wt. %) and L-lactide (46 wt. %) and also calcium stearate (ratio of copolymer to calcium stearate: 2:1), and
2^{nd} solution containing a copolymer based on ε-caprolactone, (60 wt. %), trimethylene carbonate (30 wt. %) and glycolide (10 wt. %) with or without calcium stearate.

Uncoated Dyneema® threads were used as comparative fibers.

### 3. Knot Slide

To measure the roughness of the fibers coated with the 2nd solution, the fibers were clamped into a device for measuring knot slide. The lower free ends of the fibers each had a weight of 200 g suspended from them. The measurement was based on the following parameters:

change of direction speed: 500 mm/min
test path: 150 mm, of which the first 50 mm were not measured, the measurement only being based on the subsequent 75 mm.

The measurements showed that friction was more than 50% lower for the coated fibers versus uncoated fibers.

### 4. Knot Security Factor

A surgical knot (2=1) was made in a cylinder having a circumference of about 20 cm. The knot ears were cut at a distance of 8 to 10 mm. The fibers were cut at opposite regions of the knot. The ends obtained were subsequently tightened in dynamometer clamps. The dynamometer pulled at a constant speed of 100 mm/min. The fibers had to break open in the knot in 10 test runs (knot security factor=0). If the knot slipped, an additional throw had to be added to the surgical knot. In this case, the knot geometry was then 2=1=1. The number of throws added is the knot security factor. For example, a knot geometry of 2=1=1=1 corresponds to a knot security factor of 2.

The uncoated fibers had a knot security factor of more than 6, while the coated fibers had a knot security factor of less than 4. This result illustrates that the coated fibers needed fewer knots to provide correct wound closure.

### 5. Capillarity Test

This test was used to investigate the capillarity of braided UHMWPE threads produced from coated or uncoated UHMWPE fibers. For this, the threads were vertically dipped into an aqueous solution of methylene blue (0.1% w/v). The lower ends of the threads were fixed with a weight of 2 g. The capillarity of the threads caused be solution to rise up in the threads.

In general, a thread has a medically relevant capillarity when the colored length is >1 cm after one hour.

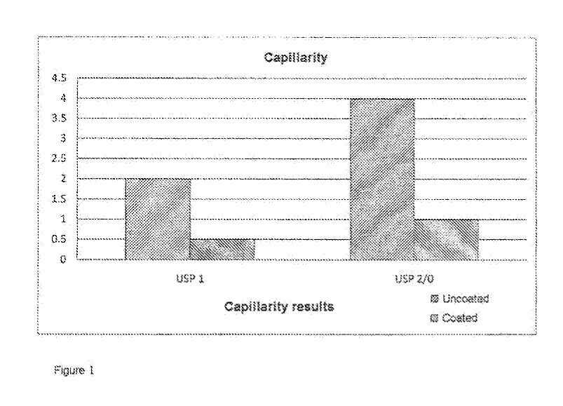

The uncoated threads had a colored length of 1.8 cm and 3.7 cm (USP1 and USP2/0 respectively) after one hour. By contrast, the coated threads had a colored length of 0.4 cm (USP1) and 0.8 cm (USP2-/0). The results are graphed in FIG. 1 for clarity.

Although the apparatus and methods have been described in connection with specific forms thereof, it will be appreciated that a wide variety of equivalents may be substituted for the specified elements described herein without departing from the spirit and scope of this disclosure as described in the appended claims.

1. A surgical thread comprising an elongate, optionally braided, thread body and a coating at least partially surrounding the thread body, wherein the thread body comprises polyethylene and/or polypropylene and the coating consists of a resorbable material and, optionally, additives.

2. The thread according to claim 1, wherein the polyethylene is selected from the group consisting of high density polyethylene (HDPE), low density polyethylene (LDPE), high molecular weight polyethylene (HMWPE), ultrahigh molecular weight polyethylene (UHMWPE), copolymers thereof and combinations thereof.

3. The thread according to claim 1, wherein the polyethylene is a crosslinked ultrahigh molecular weight polyethylene (UHMWPE).

4. The thread according to claim 1, wherein the polyethylene is an ultrahigh molecular weight polyethylene (UHMWPE) having an average molecular weight between about 10⁴ and about 10⁷ g/mol.

5. The thread according to claim 1, wherein the coating is a nontextile coating.

6. The thread according to claim 1, wherein the resorbable material is a polyhydroxyalkanoate with two or more different hydroxyalkanoate units.

7. The thread according to claim 1, wherein the resorbable material is a polymer selected from the group consisting of polyglycolide, polylactide, poly-3-hydroxybutyrate, poly-4-hydroxybutyrate, polytrimethylene carbonate, poly-para-dioxanone, poly-ε-caprolactone, copolymers thereof and mixtures thereof.

8. The thread according to claim 1, wherein the resorbable material is a copolymer comprising glycolide and lactide in a weight ratio ranging from about 9:1 to about 1:9.

9. The thread according to claim 1, wherein the additives, at least partly, are fatty acid salts, especially alkali metal and/or alkaline earth metal fatty acid salts, preferably magnesium and/or calcium fatty acid salts, especially magnesium and/or calcium stearate.

10. The thread according to claim 1, wherein the additives are in the form of fatty acid salts and account for a proportion between about 0.5 and about 5 wt. %, based on the total weight of the thread.

11. The thread according to claim 1, wherein the coating accounts for a proportion between about 1 and about 9 wt. %, based on the total weight of the thread.

12. The thread according to claim 1, wherein the coating has a layer thickness between about 1 and about 100 μm.

13. The thread according to claim 1, wherein the thread has a linear tensile strength between about 10 and about 250 N.

14. The thread according to claim 1, wherein the thread has a knot breaking strength between about 5 and about 140 N.

15. An implant in the form of a surgical suture material comprising the thread according to claim 1.

16. A process for producing the thread according to claim 1, comprising at least partially coating the polyethylene and/or polypropylene with the resorbable material and optionally the additives.

17. A process for producing the implant according to claim 15, comprising at least partially coating the polyethylene and/or polypropylene with the resorbable material and optionally the additives.
